(19) United States
(12) Patent Application Publication
(10) Pub. No.: US 2017/0215818 A1
(43) Pub. Date: Aug. 3, 2017

(54) HIGH-RESOLUTION COMPUTED TOMOGRAPHY OR C-ARM IMAGING

(71) Applicant: General Electric Company, Schenectady, NY (US)

(72) Inventors: Bruno Kristiaan Bernard De Man, Clifton Park, NY (US); Lin Fu, Niskayuna, NY (US); Mark Alan Frontera, Ballston Lake, NY (US)

(21) Appl. No.: 15/014,847

(22) Filed: Feb. 3, 2016

(51) Int. Cl. A61B 6/03; A61B 6/04; A61B 6/00

(52) U.S. Cl. CPC A61B 6/035; A61B 6/4021; A61B 6/54; A61B 6/04

(57) ABSTRACT

A high-resolution imaging approach is described. The described approach includes use of a small focal spot size and positioning of the patient offset from the center of the imaging volume. The off-center displacement is combined with a small focal spot size and with modified image reconstruction methods to provide high intrinsic spatial resolution without hardware changes to the imaging system.

### BACKGROUND

The subject matter disclosed herein relates to high resolution computed tomography imaging.

Non-invasive imaging technologies allow images of the internal structures or features of a patient to be obtained without performing an invasive procedure on the patient. In particular, such non-invasive imaging technologies rely on various physical principles, such as the differential transmission of X-rays through the target volume or the reflection of acoustic waves, to acquire data and to construct images or otherwise represent the observed internal features of the patient.

For example, in computed tomography (CT) and other X-ray based imaging technologies, X-ray radiation passes through a subject of interest, such as a human patient, and a portion of the radiation impacts a detector where the image data is collected. In digital X-ray systems a photodetector produces signals representative of the amount or intensity of radiation impacting discrete pixel regions of a detector surface. The signals may then be processed to generate an image that may be displayed for review. In the images produced by such systems, it may be possible to identify and examine the internal structures and organs within a patient's body. In CT systems a detector array, including a series of detector elements, produces similar signals through various positions as a gantry is rotationally displaced around a patient.

High spatial resolution in X-ray CT is paramount in many clinical applications. Examples of such applications include, but are not limited to, imaging of inner ear, spine, lung, and coronary artery. The intrinsic spatial resolution of a CT scanner is determined by (1) detector cell size, (2) focal spot size, and (3) sampling strategy (e.g., quarter detector offset, focal spot deflection, azimuthal blur, and so forth).

As a consequence of developments in CT hardware architecture and of conventional thinking regarding CT imaging, it is typically believed that high resolution CT imaging has to rely on fine-pitched detector hardware. However, using smaller detector cell sizes significantly increases the cost of the hardware. Moreover, the radiation dose required to image a given volume at a given noise level, increases dramatically with spatial resolution. Overall, it is challenging to design a scanner that is both low-cost and can operate with a combination of low-dose, high-spatial resolution and low-noise.

### BRIEF DESCRIPTION

In one implementation, a method for generating a high-resolution image is provided. In accordance with this implementation, a focal spot size is specified for an X-ray source of an imaging system that is less than the size of a detector cell of a detector of the imaging system. A region-of-interest of an imaged subject is positioned so the region-of-interest is offset from an iso-center of a field-of-view of the imaging system. A first set of projection data is acquired over a limited angular range that is less than 180°+α. The X-ray source moves in the limited angular range on a first side of the field-of-view containing the region of interest when acquiring the first set of projection data. One or both of a relative orientation of the region-of-interest or a relative position of the region-of interest is changed within the field-of-view. The region-of-interest remains offset from the iso-center after the change to its orientation or position. A second set of projection data is acquired over a limited angular range that is less than 180°+α. The X-ray source moves in the limited angular range on a second side of the field-of-view containing the region of interest when acquiring the second set of projection data. At least the first set of projection data and the second set of projection data are registered to generate registered projection data. The registered projection data is reconstructed to generate an image.

In a further implementation, an imaging system is provided. In accordance with this implementation, the imaging system includes a detector comprising a plurality of detector cells, and an X-ray source configured to generate X-rays at a focal spot. The imaging system also includes an imaging volume about which the detector and X-ray source rotate. The imaging volume comprises a field-of-view centered about an iso-center. The imaging system also includes a system controller configured to operate the X-ray source and detector. The system controller, during two or more scan operations: controls the X-ray source to have a focal spot size less than a detector cell size; and rotates the X-ray source and detector imaging a region-of-interest that is offset from the iso-center, wherein for each of a plurality of scans the X-ray source and detector are rotated over a respective limited angular range on a side of the field of view where the region-of-interest is offset and wherein the region-of-interest is moved or re-oriented between scans. The imaging system further includes image processing circuitry configured to reconstruct registered projection data acquired over the respective limited angular range scans.

In one implementation, a method for generating a high-resolution image is provided. In accordance with this implementation, a plurality of X-ray scan operations are performed. Each scan operation is performed over a limited angular range with respect to a field-of-view. A region-of-interest being imaged is offset from a center of the field-of-view during each scan operation and is differently positioned or oriented during each scan operation. A focal spot size from which X-rays are emitted during each scan operation is controlled so that, in one implementation, the focal spot full-width-at-half-maximum is less than 1 mm×1 mm during each scan operation. Projection data acquired during each scan operation is registered and reconstructed to generate an image.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic illustration of an embodiment of a computed tomography (CT) system configured to acquire CT images of a patient, in accordance with aspects of the present disclosure;

FIG. 2 depicts a plot illustrating the relationship between focal spot size, off-center displacement of an imaged objet, and spatial resolution, in accordance with aspects of the present disclosure;

FIG. 3 depicts an example of a process flow illustrating steps in the generation of a high-resolution image, in accordance with aspects of the present disclosure;

FIG. 4 depicts the acquisition of projection data along a limited angular range in a first quadrant, in accordance with aspects of the present disclosure;

FIG. 5 depicts the acquisition of additional projection data along a limited angular range in a second quadrant, in accordance with aspects of the present disclosure;

FIG. 6 depicts an image corresponding to a point spread function for a centered object and a conventional focal spot size;

FIG. 7 depicts an image corresponding to a point spread function for a centered object and a reduced focal spot size, in accordance with aspects of the present disclosure;

FIG. 8 depicts an image corresponding to a point spread function for an off-center object and a reduced focal spot size, in accordance with aspects of the present disclosure;

FIG. 9 depicts an image corresponding to a point spread function for an off-center object at a greater offset distance than FIG. 8 and a reduced focal spot size, in accordance with aspects of the present disclosure; and

FIG. 10 graphically plots the points spread functions of FIGS. 6-9, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

While the following discussion is generally provided in the context of medical imaging, it should be appreciated that the present techniques are not limited to such medical contexts. Indeed, the provision of examples and explanations in such a medical context is only to facilitate explanation by providing instances of real-world implementations and applications. However, the present approaches may also be utilized in other contexts, such as the non-destructive inspection of manufactured parts or goods, soil or other natural material samples (i.e., quality control or quality review applications), and/or the non-invasive inspection of packages, boxes, luggage, and so forth (i.e., security or screening applications). In general, the present approaches may be desirable in any imaging or screening context in which high-resolution images are desirable.

The present approach provides high-resolution CT imaging without changes to existing detector hardware, i.e., without reduced detector cell size. In particular, the present approach utilizes changes to the configuration of the X-ray tube (e.g., focusing voltage), the positioning of the patient (i.e., the scan protocol), the angular sampling rate (e.g., 4×, 8×, or higher), and the image reconstruction software. For example, in one implementation, the present approach places the object or part of the object being imaged off-center with respect to an iso-center of the scan volume and toward the X-ray source to achieve a high geometric magnification ratio. This off-center displacement is combined with a small focal spot size and with modified image reconstruction methods to provide high intrinsic spatial resolution without hardware changes to the imaging system. As there are no associated hardware changes, this approach allows a CT system to be switched between regular- and high-resolution modes for different clinical applications.

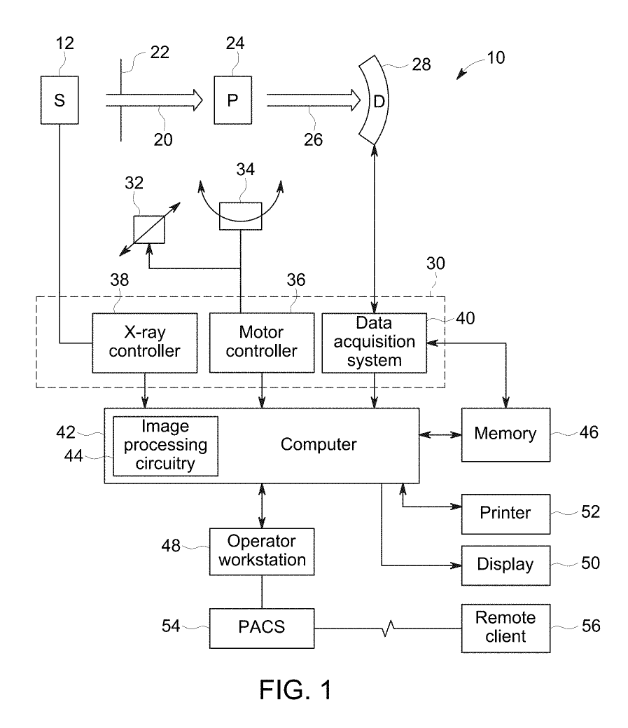

With the preceding in mind, FIG. 1 illustrates an embodiment of an imaging system 10 for acquiring and processing image data in accordance with aspects of the present disclosure. In the illustrated embodiment, system 10 is a computed tomography (CT) system designed to acquire X-ray projection data, to reconstruct the projection data into a tomographic image, and to process the image data for display and analysis. The CT imaging system 10 includes an X-ray source 12. As discussed in detail herein, the source 12 may include one or more X-ray sources, such as an X-ray tube or solid state emission structures. The X-ray source 12, in accordance with certain contemplated embodiments, is configured to emit an X-ray beam 20 from one or more emission spots (e.g., focal spots) which may be varied in size based upon the focusing voltage employed and which may correspond to X-ray emission regions on a target structure (e.g., an anode structure) impacted by a directed electron beam.

In certain implementations, the source 12 may be positioned proximate to a filter assembly or beam shaper 22 that may be used to steer the X-ray beam 20, to define the shape and/or size of a high-intensity region of the X-ray beam 20, to control or define the energy profile of the X-ray beam 20, and/or to otherwise limit incidence of the X-rays on those portions of the patient 24 not within a region-of-interest (ROI). In practice, the filter assembly or beam shaper 22 may be incorporated within the gantry between the source 12 and the imaged volume.

The X-ray beam 20 passes into a region in which the subject (e.g., a patient 24) or object of interest is positioned. As discussed herein, the position of the patient 24 (or other imaged subject or object) within the imaging volume may be determined based upon a degree of geometric magnification to be obtained, as discussed herein. The patient 24 attenuates at least a portion of the X-rays 20, resulting in attenuated X-rays 26 that impact a detector array 28 formed by a plurality of detector cells or detector elements (e.g., pixels). Each detector element produces an electrical signal that represents the intensity of the X-ray beam incident at the position of the detector element when the beam strikes the detector 28. Electrical signals are acquired and processed to generate one or more scan datasets.

A system controller 30 commands operation of the imaging system 10 to execute filtration, examination and/or calibration protocols and to process the acquired data. With respect to the X-ray source 12, the system controller 30 furnishes power, timing signals, focal size and/or spot location, and so forth, for the X-ray examination sequences. In addition, in some embodiments the X-ray controller 38 may be configured to selectively activate the source 12 such that tubes or emitters at different locations within the system 10 may be operated in synchrony with one another or independent of one another or to switch the source between different energy profiles during an imaging session.

The detector 28 is coupled to the system controller 30, which commands acquisition of the signals generated by the detector 28. In addition, the system controller 30, via a motor controller 36, may control operation of a linear positioning subsystem 32 and/or a rotational subsystem 34 used to move components of the imaging system 10 and/or the subject 24. The system controller 30 may include signal processing circuitry and associated memory circuitry. In such embodiments, the memory circuitry may store programs, routines, and/or encoded algorithms executed by the system controller 30 to operate the imaging system 10 and to process (e.g., reconstruct) the data acquired by the detector 28 in accordance with the steps and processes discussed herein. In one embodiment, the system controller 30 may be implemented as all or part of a processor-based system.

The system controller 30 may include a data acquisition system (DAS) 40. The DAS 40 receives data collected by readout electronics of the detector 28, such as sampled analog or digital signals from the detector 28. The DAS 40 may then convert and/or process the data for subsequent processing by a processor-based system, such as a computer 42. In certain implementations discussed herein, circuitry within the detector 28 may convert sampled analog signals to digital signals prior to transmission to the data acquisition system 40. The computer 42 may include or communicate with one or more non-transitory memory devices 46 that can store data processed by the computer 42, data to be processed by the computer 42, or instructions to be executed by a processor 44 of the computer 42. For example, a processor of the computer 42 may execute one or more sets of instructions stored on the memory 46, which may be a memory of the computer 42, a memory of the processor, firmware, or a similar instantiation.

The computer 42 may also be adapted to control features enabled by the system controller 30 (i.e., scanning operations and data acquisition), such as in response to commands and scanning parameters provided by an operator via an operator workstation 48. The system 10 may also include a display 50 coupled to the operator workstation 48 that allows the operator to view relevant system data, imaging parameters, raw imaging data, reconstructed data, contrast agent density maps produced in accordance with the present disclosure, and so forth. Additionally, the system 10 may include a printer 52 coupled to the operator workstation 48 and configured to print any desired measurement results. The display 50 and the printer 52 may also be connected to the computer 42 directly or via the operator workstation 48. Further, the operator workstation 48 may include or be coupled to a picture archiving and communications system (PACS) 54. PACS 54 may be coupled to a remote system 56, radiology department information system (RIS), hospital information system (HIS) or to an internal or external network, so that others at different locations can gain access to the image data.

Keeping in mind the operation of the system 10 and, specifically, the X-ray source 12 discussed above with respect to FIG. 1, examples of the operation of the imaging system 10 to generate high-resolution images are described. In one example, a number of factors may be manipulated to acquire high resolution images, the factors including, but not limited to: focal spot size (e.g., the focal spot full-width-at-half-maximum), placement of the subject or object relative to the source 12 and/or detector 28, and the reconstruction algorithms employed.

For example, in one implementation a reduced focal spot size for X-ray emission is employed. In one such implementation, the focal spot size is as small or smaller than the size of a cell of the detector 28 (e.g., equal to or less than 1 mm or 0.5 mm on a side). It is conventionally believed that, in the design of medical CT, choosing a very small focal spot yields diminishing returns in spatial resolution because the size of the detector cell becomes the major limiting factor. However, such an analysis is based on a typical geometric magnification factor (for instance, 1.7) and does not apply in the present examples where greater geometric magnification is employed. Therefore, in accordance with this approach, a small focal spot size is beneficial for the reasons discussed herein.

As will be appreciated, focal spot size of an X-ray tube is typically adjusted by configuring one or more focusing settings (e.g., focusing voltage, focusing magnet current, or other focus related parameters) of the electron beam emitted from the cathode. Such focusing setting may relate to parameters configuring or defining electro-static conditions (e.g., focusing voltage) and/or an external magnetic field (e.g., magnetic field parameters). By adjusting these focusing settings (either manually or via automated, pre-programmed routines), it is possible to reduce the focal spot size to be smaller than what have been designed for standard CT protocols and/or for a conventional CT system 10. Such focusing setting adjustments may involve changes to the system firmware and/or system calibration factors, but are straightforward to implement at the system level. As discussed in greater detail below, issues related to reduced X-ray flux (due to smaller focal spot size) are addressed below after discussion of the placement of the subject or object within the imaging system field-of-view (FOV).

In another aspect of the present approach, the region-of-interest being imaged (e.g., a region-of-interest of the patient 24 or other subject or object undergoing imaging) is placed off-center from an iso-center in the scanner field-of-view toward the source 12. For example, the region-of-interest of the patient 24 may be placed as close as possible to the focal spot of the source 12, such as at boundary of the scanner bore. In the context of a conventional CT gantry bore, the offset may take the form of one or more of positioning the region-of-interest 20 cm to 30 cm toward the edge of a 40 to 60 cm field-of-view and/or towards the edge of a 60 cm to 90 cm bore opening. This off-center positioning of the region-of-interest results in a high geometric magnification ratio (i.e., higher than what would be achieved if the region-of-interest was centered in the field-of-view) due to the region-of-interest casting a larger shadow onto the detector relative to when the region is centered. By way of example, based on the typical geometry of a medical CT imaging system, an object placed at the edge of the scanner's field-of-view can be magnified by about 3× when projected onto the detector (as the X-ray tube rotates close to the object), whereas the magnification factor is only 1.7× when the object is placed in the center of the field-of-view. The high magnification ratio substantially relaxes the need to use very small detector cell sizes to achieve high spatial resolution and, further, can be flexibly implemented “as needed”, without hardware or structural changes to the system 10. Combining high magnification (by offset of the imaged object or region) with the small focal spot size discussed above allows high spatial resolution data to be acquired.

With the preceding discussion in mind, it may be appreciated how displacement of the region being imaged within the field-of-view helps address certain flux issues raised by reducing the size of the focal spot. For example, when focal spot size is small, the flux is reduced due to thermal constraints imposed by the properties of the anode material. The flux issues associated with the small focal spot size is partly mitigated by the fact that the X-ray flux is proportional to 1/r², where r is the distance between the source 12 and the object (e.g., patient 24). Thus, in implementations where the subject or object is placed close to the X-ray source 12 to improve geometric magnification, the flux correspondingly increases due to proximity to the X-ray source 12, thus mitigating the reduction in flux attributable to small focal spot size.

Of further note, it may be noted that the angular rotational speed of the X-ray source 12 relative to the object (e.g., patient 24) is proportional to 1/r. Therefore the X-ray tube 12 also rotates faster as seen by the object when the object is placed closer to the source 12, effectively increasing the angular speed, such as up to 4× or greater than the conventional speed. As a consequence of this angular rotational speed effect, the effective exposure time is reduced by 1/r. Thus, overall, the net gain in X-ray flux is proportional to 1/r. This provides a range in which flux can be compensated to address the small focal spot size. Likewise, the gantry rotation speed can be reduced if higher X-ray flux is needed.

It may also be noted that a potential issue associated with faster rotating speed of the X-ray source 12 relative to the off-center imaged object or region-of-interest is increased azimuthal blur. However, by using a higher detector electronic sampling rate and a slower gantry rotation speed, the effect of azimuthal blur can be minimized.

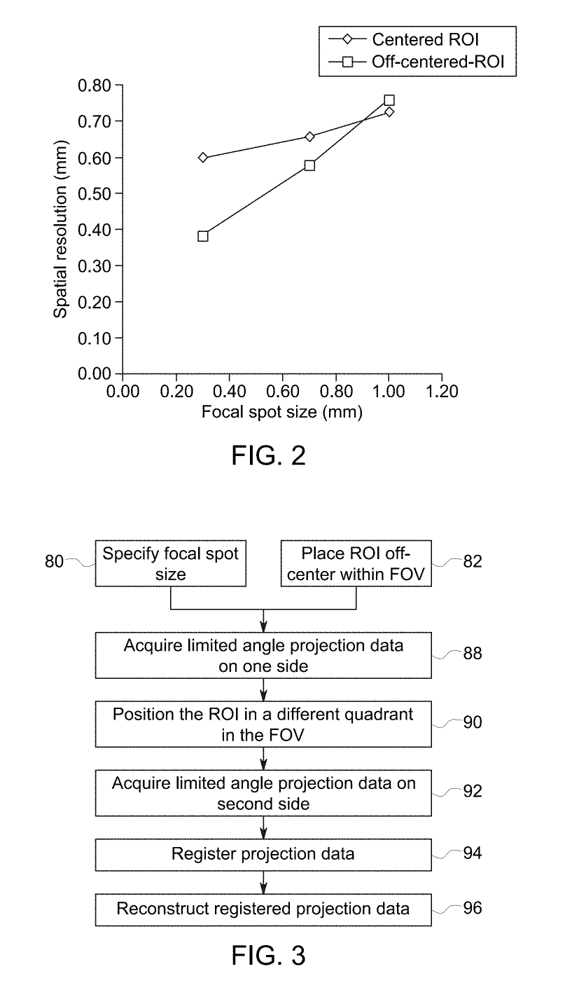

With the preceding in mind and turning to FIG. 2, an initial analysis of spatial resolution as a function of focal spot sizes and region-of-interest placement is shown. In this analysis, based on geometric considerations, system spatial resolution is plotted as a function of focal spot size for the centered (conventional) and off-centered (proposed) protocols. The off-centered placement of the region-of-interest combined with a reduction on focal spot size (e.g., focal spot full-width-at-half-maximum) from a conventional 1.0 mm×1.0 mm to a reduced 0.3 mm×0.3 mm improves spatial resolution by nearly 2× (here ˜1.7×) when compared to a conventional acquisition protocol that uses a centered region-of-interest and 1.0 mm×1.0 mm focal spot size. The plot is generated using detector cell size (i.e., pitch)=1.02 mm, source-to-isocenter (sid) distance=541 mm, source-to-detector (sdd) distance=949 mm, and a region-of-interest offset=241 mm.

With the preceding in mind, an example of a process flow in accordance with the present approach is shown in FIG. 3. As will be appreciated, the depicted sequence of steps may be varied without departing from the presently contemplated approach. For example, focal spot size and patient placement need not occur in a particular order or may occur in parallel, as shown. In accordance with this example, a focal spot size is specified (block 80) as discussed above. For example, a focal spot size may be specified that is less than the size of the cells of the detector 28 e.g., between 0.2 mm and 1.0 mm on a side or between 0.2 mm and 0.5 mm on a side. In addition, the region-of-interest (ROI) to be imaged (such as an anatomic region of a patient, a portion of an object undergoing quality review, a portion of a package being screened, and so forth) is positioned (block 82) off-center within the field-of-view (FOV) of the scanner, such as close to the boundary or edge of the scanner bore defined by the gantry covers. One or both of the positioning of the ROI and the focal spot size may be determined or adjusted to achieve the desired geometric magnification and/or X-ray flux.

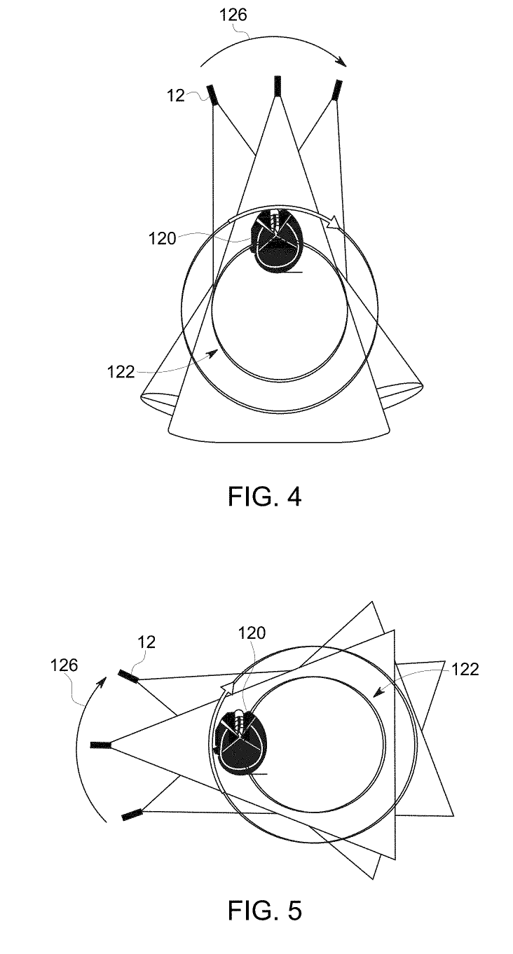

In the depicted example, projection data is acquired (block 88) over a limited angular range. This is shown in FIG. 4, where the region-of-interest 120 (here the head of a patient 24) is positioned off-center within the field-of-view 122 of the scanner while the X-ray source 12 is active over a limited angular range 126. In particular, with the off-centered placement of the region-of-interest 126, high resolution data is acquired when the X-ray source 12 rotates close to the object, but the magnification ratio will be reduced when the X-ray source rotates away from the region-of-interest 123 and the gain in spatial resolution will be lost in this situation. Thus, for the best dose efficiency, data is acquired only over a limited angular range 126 where the X-ray source 12 is close to the region-of-interest 120 and high magnification ratio results. In this manner, as shown in FIG. 4, the limited angle acquisition at one gantry or bore location obtains high resolution information in one dimension, here the horizontal direction (i.e., the x-dimension).

Such a limited angle acquisition is similar to an X-ray tomosynthesis scan, though in the present context image data is acquired continuously (i.e., for each view) within the limited angular range 126, in contrast to tomosynthesis, where acquisitions typically occur at discontinuous time and/or angular intervals (e.g., ten acquisitions over the angular range) and where the object is imaged in a single position or orientation as opposed to the two or more for the present approach (as discussed below).

In one implementation, additional projection data is acquired to provide high resolution information in a different dimension (e.g., vertical or the y-dimension in the present example). For example, with reference to FIGS. 3 and 5, the region-of-interest 120 is repositioned (block 90) to a different quadrant of the scanner field-of-view 122. Alternatively, to achieve an equivalent result, the region-of-interest 120 may remain at the same location but be rotated in place to a different orientation. The positioning and repositioning of the region-of-interest 120 can be implemented using the scanner's patient table, or specially designed holders for head or extremity for expanded range and/or precision of movement or other purposes. While the preceeding may apply in the context of a CT gantry type implementation (where the (part of the) patient is moved or turned within the bore), in other contexts a C-arm type structure may be employed. In such a C-arm context, the patient or object may remain in place and the C-arm may be repositioned so as to scan a different orientation of the patient over the limited angular range with the appropriate magnification factor.

With the preceding in mind, additional projection data is again acquired (block 92) over a limited angular range 126 in the new quadrant so as to obtain high-resolution data in a different direction (in the example of FIG. 5, the vertical or y-dimension). Though not shown, this process may also be repeated in one or more additional locations and/or orientations to acquire additional projection data. The purpose of placing the region-of-interest 120 in a different quadrant (or a different orientation) is to measure high resolution information from complementary angular ranges with adjusted magnification, as shown in FIG. 5. In this manner, by acquiring data over the complementary angular ranges with adjusted magnification, better resolution may be obtained in the horizontal and vertical (i.e., x- and y-) dimensions, as well as in the z-dimension (i.e., the direction co-axial with the rotation axis of the scanner). In addition, by only imaging over a limited angular range for each scan, radiation dose can be minimized.

With respect to the limited angular range associated with each scan, in one implementation, the limited angular range for each scan is determined so as to generate mathematically complete data (i.e., 180°+α) when aggregated with the other scans. The limited angular range for each scan may be the same as the other scans (i.e., equivalent angular ranges) or may differ between scans (i.e., some scans are over a greater angular range than others). In at least one implementation, the aggregate angular range for all scans provides 180°+α of coverage so as to be mathematically complete. For example, in an implementation where the total angular range is split evenly between scans, the limited angular range for each respective scan may be (180°+α)/2 for a scenario involving two scans, each in a different quadrant, may be (180°+α)/3 for a scenario involving two scans, each in a different quadrant, and so forth.

In the depicted process flow example, the projection data is then registered (block 94), such as using a rigid body reconstruction algorithm, so as to align the projection data acquired in separate scans for subsequent processing. By way of example, the relative position of the object (e.g., patient 24 or region-of-interest 120) may be determined based on positions of patient bed or holders, or may be estimated based on fiducial markers, or may be estimated based on image registration of the initial reconstructions. Based on one or more of these factors, the separately acquired projection scans may be registered using suitable registration algorithms.

Once the projection data is registered, the registered projection data may be reconstructed (block 96) to generate one or more high-resolution images or image volumes using the geometrically magnified projections acquired over limited angular ranges. With respect to reconstruction, a variety of approaches may be employed.

In a first approach, the two or more separately acquired projection data sets may be directly reconstructed to generate high-resolution images. By way of example, the registered projection data may be reconstructed using iterative reconstruction techniques that model the system geometry of each object (e.g., region-of-interest) placement once the relative position of the object is obtained from the registration step. For example, the known system geometry and trajectory may be modeled in a forward model used to iteratively reconstruct the high resolution image.

In a second approach, the projection data acquired over each limited angular range (i.e., incomplete measurements) may be separately reconstructed to generate low-quality images (i.e. images that are subject to limited view range artifacts and that have poor resolution in one direction and high resolution in one or two other directions). These separate low-quality images may then be combined in the Fourier (i.e., frequency) domain so as to combine complementary frequency information (e.g., complementary high-frequency information) obtained at the different spatial orientations (i.e., the different limited angular ranges). In this manner, data is merged from the partial (i.e., limited angular range) scans to generate data corresponding to a mathematically complete scan (i.e., at least 180° plus the fan angle α), in contrast to tomosynthesis. In such an implementation, the separately reconstructed images for each offset scan are recombined in Fourier space so as to generate a high-resolution final image.

In a third approach, the projection data sets acquired over the limited angular ranges are combined (i.e., rebinned) in the projection domain to form a single, high-resolution sinogram, i.e., a synthetic sinogram. This respective approach may be characterized as sinogram completion as relatively incomplete sinogram are combined (e.g., rebinned, interpolated, and so forth) to generate a complete sinogram. The combined, high-resolution sinogram is then reconstructed using conventional reconstruction algorithms (such as filtered back projection (FBP)) to generate a high-resolution final image. In certain implementations, the construction of the synthetic sinogram may involve one or both of rebinning or interpolation of the projection data so as to form a complete sinogram.

In a further approach, each scan may be over a limited angular range that is sufficient to acquire projection data corresponding to an associated sinogram that is sufficiently complete for image reconstruction. In such an approach, the respective sinograms may be separately reconstructed to generate respective images and the images can be combined in the Fourier domain (as described above). Alternatively, an optional sinogram completion step may also be provided. In such an example, the respective sinograms may be merged in a frequency dependent manner to generate a synthetic sinogram having improved frequency characteristics, which can then be reconstructed using conventional approaches to generate a high-resolution image. This respective approach may be characterized as sinogram merging, as relatively complete sinograms are merged to generate an improved sinogram having improved frequency characteristics.

With the preceding in mind, simulations were performed to assess the effectiveness of the present approach. In one such simulation, a 0.3 mm focal spot was employed to generate point spread functions for a 50 μm diameter tungsten wire embedded in a 5 cm diameter water cylinder (i.e., a small region-of-interest). The simulation was a pure two-dimensional (2D) simulation using a monochromatic 60 kV X-ray source with no simulated Poisson or acquisition system electronic noise. Quarter detector offset was disabled; focal spot profile was uniform; focal spot oversampling was 16; detector oversampling was 16; angular oversampling was 16; and the detector fill fraction was 80%.

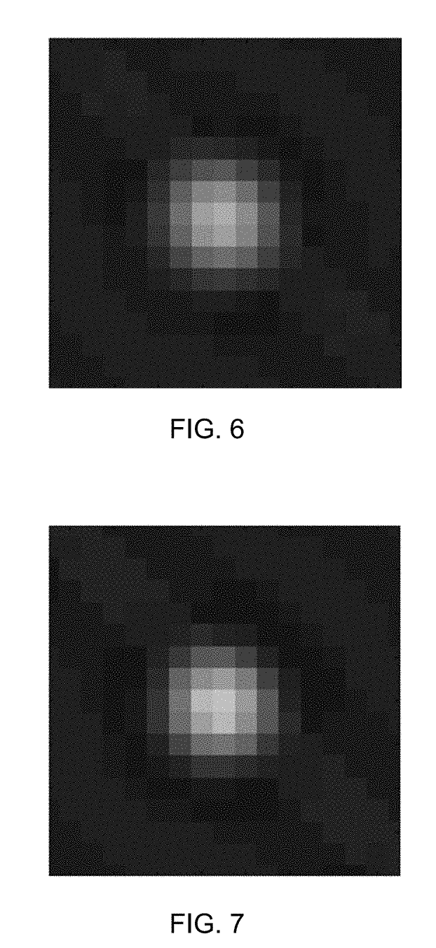

In each run, a pair of scans was performed on the phantom, each scan in a different quadrant, with focal spot size and/or phantom offset from center varied in each run. Azimuthal blur was included in the scans. A filtered backprojection (FBP) reconstruction (fan beam) was performed for each run with no beam hardening correction, an idealized reconstruction kernel, a 20 cm diameter reconstruction field-of-view, and a 1,024×1,024 reconstruction grid. Based on these simulation parameters, the results of four runs are depicted in FIGS. 6-9, with the results provided as point spread function images.

In FIG. 6 a point spread function image is depicted corresponding to a conventional approach, i.e., a centered region-of interest and a focal spot size of 1.0 mm. An intensity range of 0 to 7,000 Hounsfeld units (HU) is shown. In FIG. 7, the same parameters were employed except a focal spot size of 0.3 mm was employed.

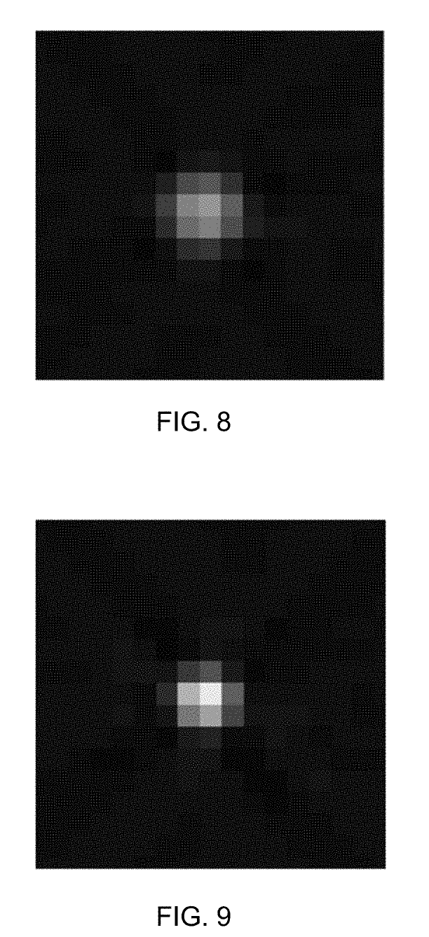

In FIG. 8, a combination of smaller focal spot size (0.3 mm) with offset of the region-of-interest was used. In particular, FIG. 8 depicts the results of a 0.3 mm focal spot size in conjunction with the region-of-interest being offset 20 cm from center toward the quadrants being imaged and having 4× angular sampling relative to the non-offset simulations. The intensity range for the image is 0 to 14,000 HU. FIG. 9 uses similar parameters to FIG. 8 except the offset is 30 cm from center toward the quadrants being imaged and angular sampling is 8× what is observed in non-offset simulations. As can be seen in viewing the sequence of images FIGS. 6-9, reduction in the focal spot size provides some observable improvement is spatial resolution (i.e., less diffusion of the pixel structure corresponding to the region-of-interest), as seen in FIGS. 6 and 7. This effect, in combination with the offset from center shown in FIGS. 8 and 9 shows markedly improved spatial resolution and reduced dispersion of the structure of interest in the image.

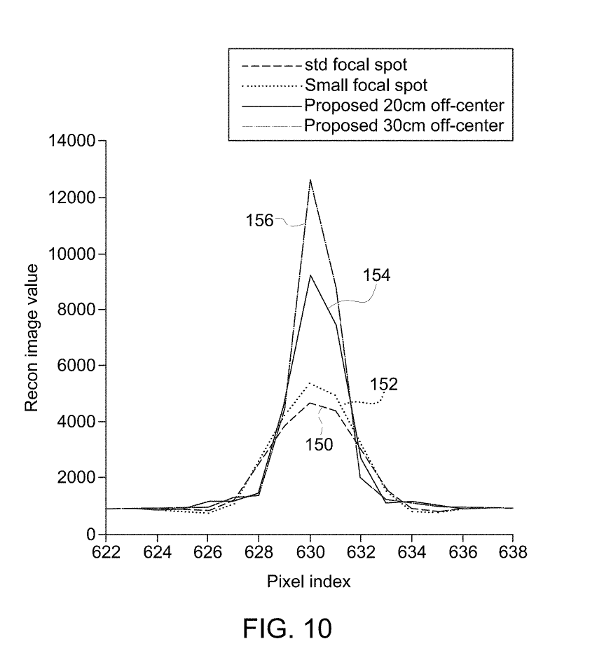

FIG. 10 graphically depicts a plot of these point spread function results for a reconstruction pixel size of 195 μm and for the object (i.e., tungsten wire) diameter of 50 μm. In FIG. 10, pixel index is shown along the x-axis while image or intensity value (in HU) is shown along the y-axis. As shown in this example, the conventional parameters (i.e., no offset and 1.0 mm focal spot size, corresponding to FIG. 6), produced (line 150) the least resolved image based on the point spread function. Spatial resolution improved in response to the small focal spot size (0.3 mm) alone (line 152, corresponding to FIG. 7) and even more so in response to the same small focal spot size in conjunction with the 20 cm off-center displacement of the region-of interest (line 154, corresponding to FIG. 8). The best spatial resolution was seen (line 156) in response to the same small focal spot size in conjunction with the 30 cm off-center displacement of the region-of interest, corresponding to FIG. 9.

Technical effects of the invention include providing an almost 2× improvement (i.e., ˜1.7 to ˜3.0) in spatial resolution without major hardware cost and without major dose penalty. Specific technical advantages include: improved resolution in the x- and z-dimensions of a scanner and flux advantage (i.e., ˜1/r). These improvements are obtained without modification or replacement of the detector. Similarly, the X-ray tube does not need to be changed or upgraded as only tube focusing setting adjustments are employed (e.g., firmware changes). Although multiple scans are performed, each scan is only over a limited angular range, thus there is no extra dose penalty for performing multiple scans. A further technical advantage is that the system may be flexibly switched between standard- and high-resolution modes.

It may be noted that the present approach may be employed in conjunction with existing technology to improve spatial resolution, such as quarter detector offset, focal spot deflection (focal spot wobbling), and sub-pixel object motion (table wobble) to increase the sampling rate of the radon space. Further, the present approach may be used in conjunction with resolution recovery and resolution boosting algorithms to further improve spatial resolution.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for generating a high-resolution image, comprising:
specifying a focal spot size for an X-ray source of an imaging system that is less than the size of a detector cell of a detector of the imaging system;
positioning a region-of-interest of an imaged subject so the region-of-interest is offset from an iso-center of a field-of-view of the imaging system;
acquiring a first set of projection data over a limited angular range that is less than 180°+α, wherein the X-ray source moves in the limited angular range on a first side of the field-of-view containing the region of interest when acquiring the first set of projection data;
changing one or both of a relative orientation of the region-of-interest or a relative position of the region-of interest within the field-of-view, wherein the region-of-interest remains offset from the iso-center after the change to its orientation or position;
acquiring a second set of projection data over the limited angular range, wherein the X-ray source moves in the limited angular range on a second side of the field-of-view containing the region of interest when acquiring the second set of projection data;
registering at least the first set of projection data and the second set of projection data to generate registered projection data; and
reconstructing the registered projection data to generate an image.

2. The method of claim 1, wherein the focal spot size is less than 1 mm.

3. The method of claim 1, wherein the focal spot size is less than half the detector cell size.

4. The method of claim 1, wherein the region-of-interest is offset 20 cm to 30 cm toward the edge of the 40 to 60 cm field-of-view or towards the edge of the 60 cm to 90 cm bore opening.

5. The method of claim 1, wherein changing one or both of a relative orientation of the region-of-interest or a relative position of the region-of interest within the field-of-view comprises moving or turning the imaged subject within a gantry of a CT imaging system.

6. The method of claim 1, wherein changing one or both of a relative orientation of the region-of-interest or a relative position of the region-of interest within the field-of-view comprises repositioning a C-arm to which the X-ray source and the detector are attached.

7. The method of claim 1, wherein the imaged subject comprises a patient, a package, a piece of baggage, a material sample, or a manufactured part.

8. The method of claim 1, wherein reconstructing the registered projection data comprises reconstructing the registered projection data using an iterative reconstruction.

9. The method of claim 1, wherein reconstructing the registered projection data comprises initially generating low-quality images and combining high-frequency information obtained at different orientations in the frequency domain to generate the image.

10. The method of claim 1, wherein reconstructing the registered projection data comprises generating a synthetic or merged sinogram and reconstructing the image from the synthetic or merged sinogram.

11. The method of claim 1, comprising:
acquiring additional sets of projection data over the limited angular range after again changing one or both of a relative orientation of the region-of-interest or a relative position of the region-of interest within the field-of-view; and
registering the additional sets of projection data with the first set of projection data and the second set of projection data to generate registered projection data.

12. The method of claim 1, wherein the limited angular ranges, when summed, correspond to angular range of at least 180° plus a fan angle α.

13. An imaging system, comprising:
a detector comprising a plurality of detector cells;
an X-ray source configured to generate X-rays at a focal spot;
an imaging volume about which the detector and X-ray source rotate, wherein the imaging volume comprises a field-of-view centered about an iso-center;
a system controller configured to operate the X-ray source and detector, wherein the system controller, during two or more scan operations:
controls the X-ray source to have a focal spot size less than a detector cell size; and
rotates the X-ray source and detector about a region-of-interest that is offset from the iso-center, wherein for each of a plurality of scans the X-ray source and detector are rotated over a respective limited angular range on a side of the field of view where the region-of-interest is offset and wherein the region-of-interest is moved or re-oriented between scans;
image processing circuitry configured to reconstruct registered projection data acquired over the respective limited angular range scans.

14. The imaging system of claim 13, wherein the focal spot size is less than 1 mm.

15. The imaging system of claim 13, wherein the imaging system comprises a computed tomography imaging system or a C-arm imaging system.

16. The imaging system of claim 13, wherein the image processing circuitry reconstructs the registered projection data using an iterative reconstruction algorithm.

17. The imaging system of claim 13, wherein the image processing circuitry reconstructs the registered projection data using complementary frequency data derived from different scans.

18. The imaging system of claim 13, wherein the image processing circuitry reconstructs the registered projection data using a synthetic or merged sinogram derived from different scans.

19. A method for generating a high-resolution image, comprising:
performing a plurality of X-ray scan operations, each scan operation is performed over a limited angular range with respect to a field-of-view, wherein a region-of-interest being imaged is offset from a center of the field-of-view during each scan operation and is differently positioned or oriented during each scan operation;
controlling a focal spot size from which X-rays are emitted during each scan operation so that the focal spot is less than 1 mm×1 mm during each scan operation;
registering and reconstructing projection data acquired during each scan operation to generate an image.

20. The method of claim 19, wherein the scan operations, when considered together, have an aggregate angular range of at least 180° plus a fan angle α.
